(19) United States
(12) Patent Application Publication
(10) Pub. No.: US 2010/0316989 A1
(43) Pub. Date: Dec. 16, 2010

(54) METHOD FOR SCREENING AN INHIBITORY AGENT OF HBV PROLIFERATION BY USING THE INTERACTION BETWEEN HBV CAPSID AND SURFACE PROTEINS BASED ON CELLULAR IMAGING

(75) Inventors: Hyesung Jeon, Seongnam-Si (KR); Soo Jin Oh, Seoul (KR); Yeon Gyu Yu, Seoul (KR); Yun-Kyoung Kim, Bucheon-Si (KR)

Correspondence Address: EDWARDS ANGELL PALMER & DODGE LLP, P.O. BOX 55874, BOSTON, MA, 02205, US

(73) Assignee: KOREA INSTITUTE OF SCIENCE AND TECHNOLOGY, Seoul (KR)

(21) Appl. No.: 12/443,740

(22) PCT Filed: Nov. 21, 2008
(86) PCT No.: PCT/KR08/06893

§ 371 Date: Mar. 31, 2009

(30) Foreign Application Priority Data
Nov. 21, 2007 (KR) 10-2007-0119060

(51) Int. Cl. C12Q 1/70; C12N 15/63; C12N 5/10

(52) U.S. Cl. 435/5; 435/320.1; 435/325; 435/362; 435/367; 435/369; 435/365

(57) ABSTRACT

The present invention relates to a method for screening an inhibitory agent of HBV proliferation by measuring the interaction (binding strength) between capsid protein and surface protein, necessary for the proliferation of HBV, by using cellular imaging, more precisely a method for measuring changes on cellular imaging caused by the interaction between a fusion protein containing PreS domain of HBV surface protein and PH (Pleckstrin homology) domain sequence and a fusion protein containing capsid protein and fluorescence protein (GFP) interacting with the said fusion protein. The method of the present invention detecting the interaction between proteins necessary for HBV proliferation at cellular level can be effectively used for the screening of a novel inhibitory agent of HBV proliferation at cellular level.

### TECHNICAL FIELD

The present invention relates to a method for screening an inhibitory agent of HBV proliferation by measuring the interaction (binding strength) between capsid protein and surface protein, necessary for the proliferation of HBV, by using cellular imaging, more precisely a method for measuring changes on cellular imaging caused by the interaction between a fusion protein containing PreS domain of HBV surface protein and PH (Pleckstrin homology) domain sequence and a fusion protein containing capsid protein and fluorescence protein (GFP) interacting with the said fusion protein.

### BACKGROUND ART

HBV (hepatitis B virus) is a member of Hepadnaviridae family which causes hepatitis B. Approximately two hundred million people are HBV carriers over the world. HBV vaccine has already been developed and widely used, but HBV treatment agents for those infected are limited to lamivudine and interferon. Lamivudine inhibits the activity of HBV DNA polymerase but it can produce resistant virus when it is administered for a long term, suggesting that the use thereof is limited. Therefore, it is required to develop diverse HBV treatment agents.

As an inhibitory agent of HBV, interferon, nucleic acid derivative or immune regulators have been developed, but the effect is in doubt. So, studies are undergoing to find out an inhibitory agent of HBV functioning to interrupt virus-receptor binding or to inhibit active proteins or polymerase thereof. Attempts have been made to develop an agent to inhibit diverse activities of HBV proteins. In HBV infected cells, the interaction of HBV capsid protein and surface protein is essential for the assembly of HBV. HBV capsid protein binds to HBV nucleic acid to form a nucleocapsid particle of 30 nm in diameter. As HBV surface proteins, three proteins, L, M, and S proteins, are biosynthesized from one gene (S gene), which are expressed in ER lipid membrane in cells and then bind specifically to the said nucleocapsid. At the same time, ER lipid membrane wraps the HBV nucleocapsid, resulting in a complete HBV particle (Volker B & Don G, PNAS USA 88:1059-1063, 1991). During while, capsid protein on the HBV nucleocapsid and the domain of amino acid residues 1-163 at N-terminal of surface protein L, particularly named as PreS, are selectively bound each other to form a HBV particle. The interaction between the two proteins can be a target of the development of a HBV proliferation inhibitory agent. To screen the interaction between HBV capsid protein and surface protein, an immuno assay using a recombinant protein has been developed (Asif-Ullah M et al., Antiviral Res 70; 85-90. 2006). This method is characterized by using a recombinant protein expressed in E. coli.

In addition to the method measuring the activity of a purified target protein, as a method measuring the bioactivity of a compound, a method to measure the activity of a target protein using a cell is actively tried during the development of a new drug. The method measuring the bioactivity of a compound in targeting cells has advantages of simultaneous detection of cellular permeability and toxicity of a target compound, making it an efficient screening method.

In HBV infected cells, HBV capsid protein and a nucleocapsid particle comprising HBV nucleic acid are specifically bound to HBV surface proteins expressed in ER membrane and as a result, active HBV particles are formed. The binding of HBV proteins is determined by the selective interaction between HBV capsid protein and PreS domain of surface protein. Therefore, if the interaction of HBV capsid protein and PreS domain of surface protein can be screened at cellular level, a compound capable of inhibiting the interaction between those proteins will be screened. So, the compound screened thereby is capable of inhibiting HBV proliferation and thus can be used as a treatment agent for HBV mediated hepatitis.

The present invention relates to a method for measuring the changes of cellular distribution of fluorescence signal generated by the interaction between a fusion protein containing PreS domain of HBV surface protein and PH sequence functioning for membrane targeting and a fusion protein containing capsid protein and fluorescence protein (GFP) interacting with the said fusion protein under fluorescent microscope. The method of the present invention is a screening method of the interaction between proteins necessary for HBV proliferation at cellular level, so that it can be effectively used for the screening of a novel inhibitory agent of HBV proliferation.

The present inventors developed a method for measuring the interaction of HBV capsid protein and surface protein at cellular level and further completed this invention by confirming that the method could be effectively used for the screening of a compound capable of inhibiting HBV proliferation by interrupting the interaction between HBV capsid protein and surface protein.

### DISCLOSURE

### Technical Problem

It is an object of the present invention to provide a method for screening an inhibitory agent of HBV proliferation efficiently by measuring the interaction between HBV capsid protein and surface protein at cellular level.

### Technical Solution

To achieve the above object, the present invention provides an expression vector containing polynucleotide encoding the fusion protein in which HBV capsid protein is linked to fluorescence protein and the other polynucleotide encoding the fusion protein in which PreS domain of HBV surface protein is linked to certain protein domain functioning for cell membrane targeting.

The present invention also provides animal cells transfected with both expression vector 1 containing polynucleotide encoding the fusion protein in which PreS domain of HBV surface protein is linked to certain protein domain functioning for cell membrane targeting and expression vector 2 containing polynucleotide encoding the fusion protein in which HBV capsid protein interacting with the said fusion protein is linked to fluorescence protein.

The present invention also provides a method for screening an inhibitory agent of HBV proliferation comprising the following steps:

1) treating candidates to the animal cells during culture;

2) taking fluorescence image of the fluorescence protein expressed in step 1) using fluorescent microscope; and

3) selecting candidates locating fluorescence image in cytoplasm.

The present invention also provides a screening kit of an inhibitory agent of HBV proliferation containing the said animal cells.

### ADVANTAGEOUS EFFECT

The method for screening the interaction of proteins necessary for HBV proliferation of the present invention can be effectively used for the screening of a novel inhibitory agent of HBV proliferation at cellular level.

### DESCRIPTION OF DRAWINGS

The application of the preferred embodiments of the present invention is best understood with reference to the accompanying drawings, wherein:

FIG. 1 is a schematic diagram illustrating the structures of the fusion protein (PreS-PH) in which PreS domain of HBV (Hepatitis B Virus) surface protein is linked to N-terminal of PH domain targeting cell membrane and the fusion protein (Capsid-GFP) in which capsid protein (HBcAg) is linked to N-terminal of green fluorescence protein (GFP).

FIG. 2 is a schematic diagram illustrating the cleavage map of the expression vector expressing PreS-PH, Capsid-GFP, and mutant PreS-PH simultaneously:

a: pHBsPH-HBcGFP;

b: pHBc-GFP; and,

c: pΔHBsPH-HBcGFP.

FIG. 3 is a schematic diagram illustrating the migration of Capsid-GFP protein from cytoplasm to cell membrane by the interaction with PreS-PH protein.

FIG. 4 is a diagram illustrating that fluorescence image of Capsid-GFP protein expressed in the cytoplasm of cell is moved to cell membrane by the interaction with PreS protein when the PreS-PH fusion proteins is co-expressed:

a: pHBc-GFP;

b: pHBsPH-HBcGFP; and,

c: pΔHBsPH-HBcGFP.

FIG. 5 is a diagram illustrating the inhibition of the interaction of capsid protein and PreS protein by PreS derived peptide.

FIG. 6 is a diagram illustrating that fluorescence image of Capsid-GFP co-expressed with PreS-PH in cells is observed in cytoplasm, resulted from the action of PreS-peptide.

### BEST MODE

Hereinafter, the present invention is described in detail.

The present invention provides an expression vector containing polynucleotide encoding the fusion protein in which HBV capsid protein is linked to fluorescence protein and the other polynucleotide encoding the fusion protein in which PreS domain of HBV surface protein is linked to certain protein domain functioning for cell membrane targeting.

The said expression vector can be prepared by inserting a polynucleotide encoding the fusion protein comprising HBV capsid protein and fluorescence protein and a polynucleotide encoding the fusion protein comprising PreS domain of HBV surface protein and cell membrane targeting protein domain into the basic vector. The basic vector used in this invention can be any vector applicable in animal cell transfection which is preferably selected from the group consisting of pBud-CE 4.1, pcDNA3.1, pcDNA4 and pEF6p, but not always limited thereto. In a preferred embodiment of the present invention, pBud-CE4.1 was used.

The capsid protein herein can be full length capsid protein or a fragment capable of interacting with the surface protein. In a preferred embodiment of the invention, HBV capsid protein having the amino acid sequence without pro sequence, represented by SEQ. ID. NO: 19 (amino acids 30-214), was used.

The fluorescence protein herein is exemplified by green fluorescence protein (GFP), red fluorescence protein (RFP), blue fluorescence protein (BFP), yellow fluorescence protein (YFP), cyan fluorescence protein (CFP) and enhanced green fluorescence protein (EGFP), but not always limited thereto and in a preferred embodiment of the present invention, GFP was used.

The surface protein herein can be full length PreS domain or a fragment of PreS domain capable of interacting with capsid protein or PreS domain of HBV surface protein excluding the domain ranging from amino acid residue #93 to amino acid residue #117 capable of interacting core part in capsid protein. In a preferred embodiment of the present invention, PreS domain having the amino acid sequence of HBV surface protein capable of interacting with capsid protein, represented by SEQ. ID. NO: 20, was used. In another preferred embodiment of the present invention, PreS domain excluding the domain from amino acid #93 to amino acid #117 interacting with core of capsid protein was used.

The cell membrane targeting protein domain in step 1) is exemplified by PH (Pleckstrin homology) domain of PLC-6 (phospholipase C delta) (Genebank ID: 241276, amino acids 2-175), FYVE (No full name) domain of EEA1 (early endosome antigene1) (Genebank ID: L40157, amino acids 1352-1410), PHD (Prolyl-hydroxylase) domain of ING2 (Inhibitor of growth2) (Genebank ID: NM_{—}001564, amino acids 212-261), C2 (calcium/lipid-binding) domain of protein kinase C (Genebank ID: NM002737, amino acids 172-260) and SEC14 (S. cerevisiae phosphatidylinositol transfer protein homology) domain of guanine nucleotide exchange factor DBS (Genebank ID: AB 116074, amino acids 90-236), but not always limited thereto. In a preferred embodiment of the present invention, PH domain of PLC-δ was used.

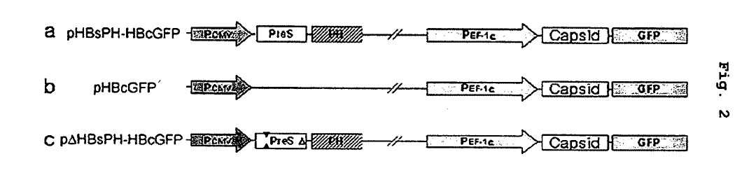

The capsid-GFP fusion protein has the amino acid sequence represented by SEQ. ID. NO: 4 (see FIG. 2b). The amino acid sequence represented by SEQ. ID. NO: 4 is characteristically coded by the nucleotide sequence represented by SEQ. ID. NO: 3.

The fusion protein comprising PreS domain of HBV surface protein and cell membrane targeting protein domain has the amino acid sequence represented by SEQ. ID. NO: 2 (see FIG. 2a). The amino acid sequence represented by SEQ. ID. NO: 2 is characteristically coded by the nucleotide sequence represented by SEQ. ID. NO: 1.

In another preferred embodiment of the present invention, the PreS domain of HBV surface protein can be substituted with another PreS domain excluding the domain ranging from amino acid #93 to amino acid #117 interacting core in capsid protein. That is, the domain from amino acid #93 to amino acid #117 interacting core region is eliminated from PreS domain to which cell membrane targeting protein domain is conjugated, resulting in the fusion protein having the amino acid sequence represented by SEQ. ID. NO: 6 (see FIG. 2c). The amino acid sequence represented by SEQ. ID. NO: 6 is characteristically coded by the nucleotide sequence represented by SEQ. ID. NO: 5.

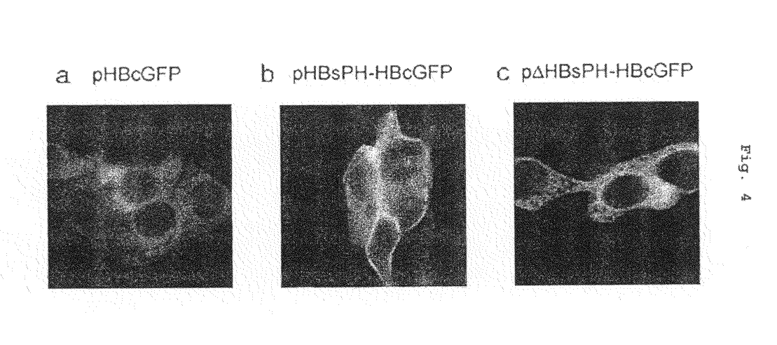

The interaction of capsid protein and surface protein can be clearly detected when the expression vector (pΔHBsPH-HBcGFP, see FIG. 4c) co-expressing the fusion protein in which HBV capsid protein is linked to fluorescence protein and the fusion protein in which PreS domain excluding the amino acid domain from amino acid #93 to amino acid #117 interacting with core of capsid protein was used, compared to when the expression vector (pHBsPH-HBcGFP, see FIG. 4b) co-expressing the fusion protein in which HBV capsid protein is linked to fluorescence protein and the fusion protein in which PreS domain of HBV surface protein is linked to cell membrane targeting protein domain was used, because the interaction of core in capsid protein with surface protein is eliminated when the former was used (see FIG. 4).

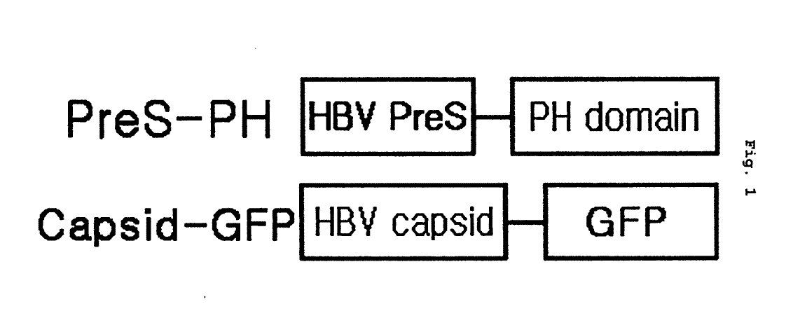

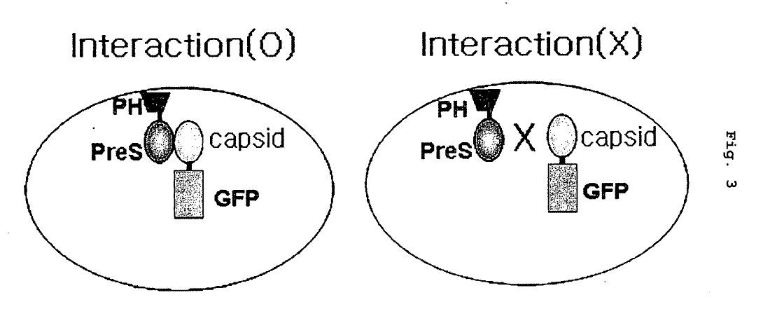

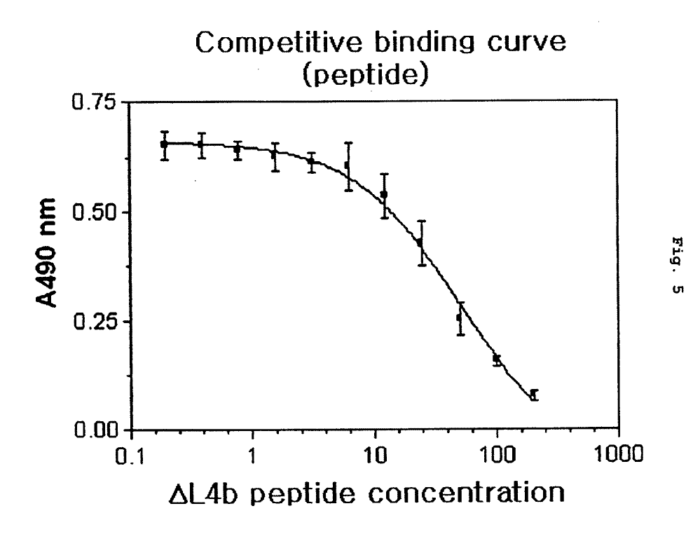

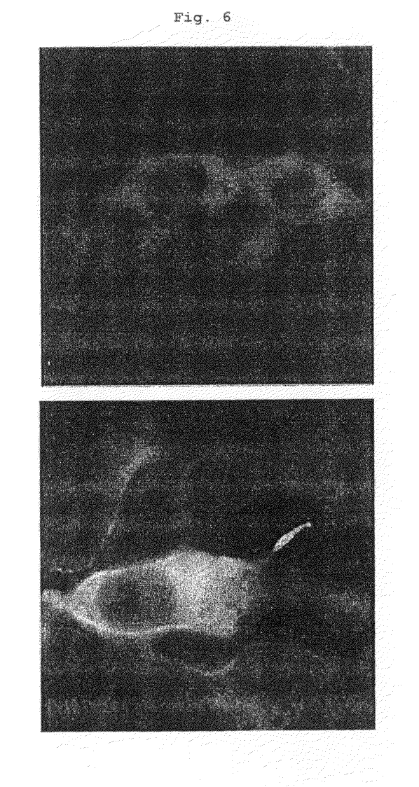

The capsid protein of the present invention can be conjugated with fluorescence protein and PreS domain of surface protein can be conjugated with cell membrane targeting protein (see FIG. 1). The interaction of fluorescence protein conjugated capsid protein and cell membrane targeting protein conjugated PreS domain makes the fluorescence protein conjugated capsid protein to move from cytoplasm to cell membrane (see FIG. 3). The present inventors confirmed that the interaction of capsid protein and PreS domain was inhibited by PreS derived peptide and fluorescence image of the fusion protein comprising capsid protein-fluorescence protein was located not in cell membrane but in cytoplasm (see FIGS. 5 and 6). Accordingly, the present inventors confirmed that fluorescence protein conjugated capsid protein specifically interacted with cell membrane targeting protein conjugated PreS domain of HBV surface protein.

The present invention also provides animal cells transfected with the above expression vector.

The animal cells herein can be exemplified by HEK293T, COS7, HeLa and CHO cells, but not always limited thereto and in a preferred embodiment of the present invention HEK293T cells were used.

The present invention further provides animal cells transfected with both expression vector 1 containing polynucleotide encoding the fusion protein (PreS-PH) in which PreS domain of HBV surface protein is linked to certain protein domain functioning for cell membrane targeting and expression vector 2 containing polynucleotide encoding the fusion protein (Capsid-GFP) in which HBV capsid protein interacting with the said fusion protein is linked to fluorescence protein.

In a preferred embodiment of the present invention, PreS-PH and Capsid-GFP are cloned into different vectors having different origins but co-expressed in animal cells together by co-transfection.

The present invention also provides a screening method of an inhibitory agent of HBV proliferation comprising the following steps:

1) treating candidates to the animal cells during culture;

2) taking fluorescence image of the fluorescence protein expressed in step 1) using fluorescent microscope; and

3) selecting candidates locating fluorescence image in cytoplasm.

In addition, the present invention provides a screening kit of an inhibitory agent of HBV proliferation containing the said animal cells.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Construction of Expression Vector

<1-1>Construction of pCapsid-GFP

PCR was performed using pHBcAg (Choi K J et al., Biochem Biophys Res Commun 319:959-66, 2004) containing HBV capsid protein excluding pro sequence (amino acids 30-214, SEQ. ID. NO: 19) as a template with the primers represented by SEQ. ID. NO: 7 and NO: 8 as follows: at 95° C. for 1 minute, at 55° C. for 30 seconds, and at 72° C. for 1 minute (25 cycles). The amplified product was digested with SalI and KpnI, and then inserted into pEGFP—N1 vector (Clontech) digested with the same restriction enzymes by using T4 DNA ligase. As a result, pCapsid-GFP containing capsid-GFP was constructed. PCR was performed using the above DNA as a template with the primers represented by SEQ. ID. NO: 9 and NO: 10 to produce capsid-GFP DNA fragments containing the DNA restriction enzymes, NotI and XhoI. The synthesized capsid-GFP and pBud-CE 4.1 (Stratagene, USA) were digested with NotI and XhoI, followed by ligation using T4 DNA to construct pHBc-GFP (FIG. 2b). The DNA was sequenced to confirm whether the capsid-GFP DNA represented by SEQ. ID. NO: 3 encoding the amino acid sequence represented by SEQ. ID. NO: 4 was successfully inserted.

### <1-2>Construction of PreS-PH

PCR was performed using rat cDNA as a template with the primers represented by SEQ. ID. NO: 11 and NO: 12 to synthesize PH domain (Genebank ID: 241276, amino acids 2-175) of PLC-δ (phospholipase C delta) as follows: at 95° C. for 1 minute, at 55° C. for 30 seconds, and at 72° C. for 1 minute (25 cycles). PCR was also performed using pTrx-PreS (Choi K J et al., Biochem Biophys Res Commun 319:959-66, 2004) as a template with the primers represented by SEQ. ID. NO: 13 and NO: 14 to synthesize PreS domain (amino acids 1-163, SEQ. ID. NO: 20) of HBV surface protein as follows: at 95° C. for 1 minute, at 55° C. for 30 seconds, and at 72° C. for 1 minute (25 cycles). PCR was also performed using the DNA encoding PreS and the DNA encoding PH as templates with the primers represented by SEQ. ID. NO: 15 and NO: 16 to synthesize PreS-PH DNA as follows: at 95° C. for 1 minute, at 55° C. for 30 seconds, and at 72° C. for 1 minute (25 cycles). At this time, the two templates were overlapped. The amplified DNA was digested with HindIII and XbaI, followed by ligation to pHBc-GFP digested with the same restriction enzymes by using T4 DNA. As a result, pHBsPH-HBcGFP (FIG. 2b) containing DNA having the nucleotide sequence represented by SEQ. ID. NO: 1 encoding the amino acid sequence represented by SEQ. ID. NO: 2 was constructed. The DNA was sequenced to confirm whether the amplified DNA was successfully inserted.

PCR was also performed using pHBsPH-HBcGFP without the domain ranging from amino acid #93 to amino acid #117 interacting with core protein as a template with the primers represented by SEQ. ID. NO: 17 and NO: 18 to synthesize DNA having the nucleotide sequence represented by SEQ. ID. NO: 5 encoding the amino acid sequence represented by SEQ. ID. NO: 6 as follows: at 95° C. for 30 seconds, at 55° C. for 1 minute, and at 68° C. for 7 minutes (18 cycles). The domain other than non-methylated DNA was eliminated by treating DpnI, the restriction enzyme recognizing methylated adenine. pΔHBsPH-HBcGFP (FIG. 2c) was confirmed by nucleotide sequencing.

### Example 2

### Animal Cell Transformation

### <2-1>Animal Cell Culture

HEK293T cells were cultured in DMEM (Dulbecco's modified Eagle's medium; Gibco, USA) supplemented with 10% FBS (fetal bovine serum) in a 5% CO₂, 37° C. incubator. The cells were sub-cultured when the density reached 90% and thus the density was adjusted to 25%, which was maintained until transformation.

### <2-2>Animal Cell Transformation

The HEK293T cells cultured in Example <2-1> were sub-cultured, followed by further culture in a 6 well plate with cover glass. The cells were cultured until the density reached 40-60%. Then, the cells were transfected with pHBc-GFP, pHBsPH-HBcGFP or pΔHBsPH-HBcGFP by using Lipofectamine (Invitrogen, USA) and PLUS reagent (Invitrogen, USA). The transfected cells were cultured for 48 hours to produce protein.

### Example 3

### Inhibition of Interaction Between Capsid Protein and PreS Protein by PreS Derived Peptide

It was investigated whether PreS derived peptide (ΔL4b peptide; SEQ. ID. NO: 21: RQPTPISPPLRDSHPQAMQWNS; Peptron, Inc., Korea) could inhibit the interaction between PreS protein and capsid protein.

Thioredoxin conjugated PreS protein purified from E. coli transfected with pTrx-PreS (Choi K J et al., Biochem Biophys Res Commun 319:959-66, 2004) was dissolved in buffer-A (50 mM sodium phosphate, 0.15M NaCl, pH 8.0) at the concentration of 10 g/ml. 100 μl of the mixed solution was loaded in each well of a 96 well plate (CoStar, USA), followed by incubation for one hour at room temperature for fixation. The plate was blocked with buffer-A containing 5% (w/v) skim milk, to which different concentrations of serially diluted ΔL4b peptide and capsid protein purified from E. coli transfected with pHBcAg (Choi K J et al., Biochem Biophys Res Commun 319:959-66, 2004) were added (final conc.: 0.4 mM). Incubation was continued for 60 minutes at room temperature, followed by washing 6 times with PBS-T buffer (50 mM sodium phosphate, 0.15 M NaCl, pH 7.4 and 0.1% Tween-20) (300 a/well). The plate was incubated with anti-HBcAg antibody (1:2000, Cat. No. K0112162, KOMA biotechnology, Korea) for one hour (100 μl/well), to which HRP labeled anti-rabbit secondary antibody (1:2000, Sigma, USA) was added, followed by further incubation for one more hour. The plate was washed 6 times with PBS-T buffer, followed by color development using OPD solution (100 μl/well, dissolved in peroxide substrate buffer the concentration of 1 mg/m). The reaction was terminated by adding 2.5 M sulfuric acid (100 pt/well). Then, OD₄₉₀ was measured by using multiplate reader (Spectra Max340 spectrometer, Molecular Devices Corp., USA).

As a result, it was confirmed that the PreS derived peptide inhibited the interaction between capsid protein and PreS protein dose-dependently (FIG. 5).

### Example 4

### Cell Imaging Using Fluorescent Microscope

### <4-1>Measurement of Interaction

Cover glass was recovered from the plate where the HEK293T cells transfected with pHBc-GFP, pHBsPH-HBcGFP or pΔHBsPH-HBcGFP were growing, and washed with ice-cold PBS. The cover glass was attached onto slide glass for observation under microscope. The cells were observed through filter (490±20/528±38 nm, excitation/emission) to observe GFP under fluorescent microscope.

As a result, GFP fluorescence image was observed in cytoplasm in the cells transfected with pHBc-GFP or pΔHBsPH-HBcGFP, while GFP fluorescence image was observed in cell membrane in the cells transfected with pHBsPH-HBcGFP (FIG. 4). The fluorescence image in the cells transfected with pΔHBsPH-HBcGFP confirmed the importance of interaction between the core in capsid protein and the surface protein.

### <4-2>Measurement of Inhibition of Interaction

When the HEK293T cells transfected with pHBc-GFP, pHBsPH-HBcGFP or pΔHBsPH-HBcGFP were reached 60% of confluence, PreS derived peptide (ΔL4b peptide; SEQ. ID. NO: 21) inhibiting the interaction between PreS and capsid protein was treated at the concentration of 50 μM thereto. Cover glass was recovered from the plate where the transfected HEK293T cells were growing, and washed with ice-cold PBS. The cover glass was attached onto slide glass for observation under microscope. The cells were observed through filter (490±20/528±38 nm, excitation/emission) to observe GFP under fluorescent microscope.

As a result, it was confirmed that the fluorescence image of fusion protein conjugated with GFP was located not in cell membrane but in cytoplasm of cells, suggesting that the interaction was inhibited (FIG. 6).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A expression vector comprising expression vector 1 containing a first polynucleotide encoding a first fusion protein in which PreS domain of HBV surface protein is linked to certain protein domain functioning for cell membrane targeting a HBV capsid protein domain is linked to a fluorescence protein and expression vector 2 containing a polynucleotide encoding a second fusion protein in which a PreS domain of HBV surface protein is linked to a protein domain functioning for cell membrane targeting.

2. The expression vector according to claim 1, wherein the HBV capsid protein domain has the amino acid sequence represented by SEQ. ID. NO: 19 of HBV capsid protein except pro sequence (amino acids nos. 30-214).

3. The expression vector according to claim 1, wherein the fluorescence protein is selected from the group consisting of green fluorescence protein (GFP), red fluorescence protein (RFP), blue fluorescence protein (BFP), yellow fluorescence protein (YFP), cyan fluorescence protein (CFP) and enhanced green fluorescence protein (EGFP).

4. The expression vector according to claim 1, wherein the PreS domain of the surface protein has the amino acid sequence represented by SEQ. ID. NO: 20.

5. The expression vector according to claim 1, wherein the PreS domain of the surface protein is deficient in the part ranging from amino acid no. 93 to amino acid no. 117 of the sequence represented by SEQ. ID. NO: 20.

6. The expression vector according to claim 1, wherein the cell membrane targeting protein domain is selected from the group consisting of PH (Pleckstrin homology) domain of PLC-δ (phospholipase C delta) (Genebank ID: 241276, amino acid nos. 2-175), FYVE domain of EEA1 (early endosome antigene1) (Genebank ID: L40157, amino acid nos. 1352-1410), PHD (Prolyl-hydroxylase) domain of ING2 (Inhibitor of growth2) (Genebank ID: NM_{—}001564, amino acid nos. 212-261), C2 (calcium/lipid-binding) domain of protein kinase C (Genebank ID: NM002737, amino acid nos. 172-260) and SEC14 (S. cerevisiae phosphatidylinositol transfer protein homology) domain of guanine nucleotide exchange factor DBS (Genebank ID: AB_{—}116074, amino acid nos. 90-236).

7. The expression vector according to claim 1, wherein the first fusion protein has the amino acid sequence represented by SEQ. ID. NO: 4.

8. The expression vector according to claim 1, wherein the second fusion protein has the amino acid sequence represented by SEQ. ID. NO: 2.

9. The expression vector according to claim 1, wherein the second fusion protein's PreS domain is deficient in the part ranging from amino acid no. 93 to amino acid no. 117 and the second fusion protein has the amino acid sequence represented by SEQ. ID. NO: 6.

10. An animal cell transfected with the expression vector of claim 1, wherein said expression vector 1 comprises a first polynucleotide encoding a first fusion protein in which a HBV capsid protein domain is linked to a fluorescence protein and said expression vector 2 comprises a polynucleotide encoding a second fusion protein in which a PreS domain of HBV surface protein is linked to a protein domain functioning for cell membrane targeting.

11. The animal cell according to claim 10, wherein the cell is selected from the group consisting of HEK293T, COS7, HeLa and CHO.

12. A method for screening an inhibitory agent of HBV proliferation comprising the following steps:
1) treating candidates with the animal cell of claim 10 during culture;
2) taking fluorescence image of the fluorescence protein expressed in step 1) using fluorescent microscope; and
3) selecting candidates locating fluorescence image in cytoplasm.

13. A screening kit of an inhibitory agent of HBV proliferation containing the animal cell of claim 10.


SEQUENCE LISTING (21 sequences)

SEQ ID NO: 1 - DNA, 1044 bp, Artificial Sequence
  PreS-PH nucleotide sequence
atggggacgaatctttctgttcccaatcctctgggattctttcccgatcaccagttggaccctgcgttcggagccaactcaaacaatccagattgggacttcaaccccaacaaggatcactggccagaggcgaatcaggtaggagcgggagcattcgggccagggttcaccccaccacacggcggtcttttggggtggagccctcaggctcagggcatattgacagcagtgccagcagcgcctcctcctgcctccaccaatcggcagtcaggaagacagcctactcccatctctccacctctaagagacagtcatcctcaggccatgcagtggaattccacaacattccaccaagctctgctagatcccagagtgaggggcctatattttcctgctggtggctccagttccggaacagtaaaccctgttccgactactgcctctcccatatcgtcaatcttctcgaggactggggaccctgcaccgaaccgtggacagggaaacagcgatgcatctgtggactcgggtagggacttcctgaccctgcacgggctccaggatgacccggaccttcaggcccttctgaagggcagccagcttctgaaggtgaagtccagctcgtggcgtagggaacgcttctacaagctacaggaggactgcaagaccatctggcaggaatctcgaaaggtcatgaggtccccggagtcgcagctgttctccatcgaggacattcaggaggtacggatgggacaccgcacagaaggcctggagaagtttgcccgagacatccccgaggatcgatgcttctccattgtcttcaaggaccagcgcaacaccctagacctcattgccccatcaccagctgacgctcagcactgggtgcagggcctgcgcaagatcatccaccactccggctccatggaccagcggcagaagctgcagcactggattcactcctgcttgcgaaaggctgataaaaacaaggcaaacaagatgaacttcaaggagctgaaggacttcctgaaggagctcaacatccagtaa

SEQ ID NO: 2 - protein, 347 aa, Artificial Sequence
  PreS-PH amino acid sequence
MGTNLSVPNPLGFFPDHQLDPAFGANSNNPDWDFNPNKDHWPEANQVGAGAFGPGFTPPHGGLLGWSPQAQGILTAVPAAPPPASTNRQSGRQPTPISPPLRDSHPQAMQWNSTTFHQALLDPRVRGLYFPAGGSSSGTVNPVPTTASPISSIFSRTGDPAPNRGQGNSDASVDSGRDFLTLHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQHWIHSCLRKADKNKANKMNFKELKDFLKELNIQ

SEQ ID NO: 3 - DNA, 1399 bp, Artificial Sequence
  Capsid-GFP nucleotide sequence
atgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttttttccttccgtcagagatctcctagacaccgcctcagctctgtatcgggaagccttagagtctcctgagcattgctcacctcaccatactgcactcaggcaagcaattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgtacggtaccgcgggcccgggatcccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - protein, 466 aa, Artificial Sequence
  Capsid-GFP amino acid sequence
MQLFHLCLIISCTCPTVQASKLCLGWLWGMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCTVPRARDPTGRHHGEQGRGAVHRGGAHPGRAGRRRKRPQVQRVRRGRGRCHLRQADPEVHLHHRQAARALAHPRDHPDLRRAVLQPLPRPHEAARLLQVRHARRLRPGAHHLLQGRRQLQDPRRGEVRGRHPGEPHRAEGHRLQGGRQHPGAQAGVQLQQPQRLYHGRQAEERHQGELQDPPQHRGRQRAARRPLPAEHPHRRRPRAAARQPLPEHPVRPEQRPQREARSHGPAGVRDRRRDHSRHGRAVQV

SEQ ID NO: 5 - DNA, 969 bp, Artificial Sequence
  deleted PreS-PH nucleotide sequence
atggggacgaatctttctgttcccaatcctctgggattctttcccgatcaccagttggaccctgcgttcggagccaactcaaacaatccagattgggacttcaaccccaacaaggatcactggccagaggcgaatcaggtaggagcgggagcattcgggccagggttcaccccaccacacggcggtcttttggggtggagccctcaggctcagggcatattgacagcagtgccagcagcgcctcctcctgcctccaccaatcggcagtcaggaagacaagctctgctagatcccagagtgaggggcctatattttcctgctggtggctccagttccggaacagtaaaccctgttccgactactgcctctcccatatcgtcaatcttctcgaggactggggaccctgcaccgaaccgtggacagggaaacagcgatgcatctgtggactcgggtagggacttcctgaccctgcacgggctccaggatgacccggaccttcaggcccttctgaagggcagccagcttctgaaggtgaagtccagctcgtggcgtagggaacgcttctacaagctacaggaggactgcaagaccatctggcaggaatctcgaaaggtcatgaggtccccggagtcgcagctgttctccatcgaggacattcaggaggtacggatgggacaccgcacagaaggcctggagaagtttgcccgagacatccccgaggatcgatgcttctccattgtcttcaaggaccagcgcaacaccctagacctcattgccccatcaccagctgacgctcagcactgggtgcagggcctgcgcaagatcatccaccactccggctccatggaccagcggcagaagctgcagcactggattcactcctgcttgcgaaaggctgataaaaacaaggcaaacaagatgaacttcaaggagctgaaggacttcctgaaggagctcaacatccagtaa

SEQ ID NO: 6 - protein, 322 aa, Artificial Sequence
  deleted PreS-PH amino acid sequence
MGTNLSVPNPLGFFPDHQLDPAFGANSNNPDWDFNPNKDHWPEANQVGAGAFGPGFTPPHGGLLGWSPQAQGILTAVPAAPPPASTNRQSGRQALLDPRVRGLYFPAGGSSSGTVNPVPTTASPISSIFSRTGDPAPNRGQGNSDASVDSGRDFLTLHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQHWIHSCLRKADKNKANKMNFKELKDFLKELNIQ

SEQ ID NO: 7 - DNA, 33 bp, Artificial Sequence
  HBV Capsid forward primer
gccgagctcgccaccatgcaactttttcacctc

SEQ ID NO: 8 - DNA, 28 bp, Artificial Sequence
  HBV Capsid reverse primer
ggggtaccgtataacattgagattcccg

SEQ ID NO: 9 - DNA, 47 bp, Artificial Sequence
  Capsid-GFP forward primer
aaggaaaaagcggccgcgcgctcgccaccatgcaactttttcacctc

SEQ ID NO: 10 - DNA, 33 bp, Artificial Sequence
  Capsid-GFP reverse primer
ccgctcgagttacttgtacagctcgtcccatga

SEQ ID NO: 11 - DNA, 28 bp, Artificial Sequence
  Mouse PLC delta forward primer
gactcgggtagggacttcctgaccctgc

SEQ ID NO: 12 - DNA, 28 bp, Artificial Sequence
  Mouse PLC delta reverse primer
ttactggatgttgagctccttcaggaag

SEQ ID NO: 13 - DNA, 34 bp, Artificial Sequence
  HBV PreS forward primer
gccgagctcgccaccatggggacgaatctttctg

SEQ ID NO: 14 - DNA, 35 bp, Artificial Sequence
  HBV PreS reverse primer
aaggaaaaagcggccgcttactggatgttgagctc

SEQ ID NO: 15 - DNA, 40 bp, Artificial Sequence
  PreS-PH forward primer
cccaagctttgcgctcgccaccatgcaactttttcacctc

SEQ ID NO: 16 - DNA, 26 bp, Artificial Sequence
  PreS-PH reverse primer
gctctagattactggatgttgagctc

SEQ ID NO: 17 - DNA, 30 bp, Artificial Sequence
  deleted PreS-PH foward primer
ggcagtcaggaagacaagctctgctagatc

SEQ ID NO: 18 - DNA, 30 bp, Artificial Sequence
  deleted PreS-PH reverse primer
gatctagcagagcttgtcttcctgactgcc

SEQ ID NO: 19 - protein, 214 aa, Artificial Sequence
  HBV capsid protein amino acid sequence
MQLFHLCLIISCTCPTVQASKLCLGWLWGMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 20 - protein, 163 aa, Artificial Sequence
  HBV PreS protein amino acid sequence
MGTNLSVPNPLGFFPDHQLDPAFGANSNNPDWDFNPNKDHWPEANQVGAGAFGPGFTPPHGGLLGWSPQAQGILTAVPAAPPPASTNRQSGRQPTPISPPLRDSHPQAMQWNSTTFHQALLDPRVRGLYFPAGGSSSGTVNPVPTTASPISSIFSRTGDPAPN

SEQ ID NO: 21 - protein, 22 aa, Artificial Sequence
  deleted L4b peptide
RQPTPISPPLRDSHPQAMQWNS